(19) United States
(12) Patent Application Publication
(10) Pub. No.: US 2024/0043907 A1
(43) Pub. Date: Feb. 8, 2024

(54) MRNA ANALYSIS USING RESTRICTION ENZYMES

(71) Applicant: Waters Technologies Corporation, Milford, MA (US)

(72) Inventors: Martin Gilar, Franklin, MA (US); Catalin Doneanu, Franklin, MA (US); Matthew A. Lauber, North Smithfield, RI (US); Mame Maissa Gaye, Cambridge, MA (US)

(73) Assignee: Waters Technologies Corporation, Milford, MA (US)

(21) Appl. No.: 18/366,149

(22) Filed: Aug. 7, 2023

Related U.S. Application Data
(60) Provisional application No. 63/395,978, filed on Aug. 8, 2022.

(51) Int. Cl. C12Q 1/6806; G16B 30/00

(52) U.S. Cl. CPC C12Q 1/6806; G16B 30/00

(57) ABSTRACT

The present disclosure describes methods, kits, and systems for digesting polyribonucleotides. The method involves selectively forming oligonucleotide (e.g., DNA:RNA or RNA:RNA) duplexes with single-stranded target RNA and then using sequence-specific nucleases that only act on RNA within duplexes to selectively cleave the target RNA into smaller fragments. Additional sequence-specific ribonucleases may be used to provide additional cuts of the target RNA at predetermined sites. By forming duplexes to increase the availability of nucleases that may be applied to cleave the single-stranded target RNA and selectively control where the target RNA is cleaved, the target RNA may be digested into fragments within controllable size ranges that are optimal for polynucletide analysis, such as by liquid chromatography and mass spectrometry.

### CROSS-REFERENCE TO RELATED APPLICATIONS

This application claims the benefit of and priority to U.S. Provisional Patent Application No. 63/395,978 filed on Aug. 8, 2022 titled “mRNA ANALYSIS USING RESTRICTION ENZYMES,” the entire contents of which is hereby incorporated by reference in its entirety.

### FIELD OF THE TECHNOLOGY

The present disclosure relates to the use of enzymes that cleave RNA within duplexes to selectively cleave large RNA molecules into fragments of predetermined sizes for polynucleotide analysis.

### BACKGROUND

mRNA is being used as a new therapeutic modality, including in vaccines and protein replacement therapy. During the enzymatic manufacturing process of mRNA therapeutics, incomplete mRNA products are generated in conjunction with other potential impurities such as double-stranded RNA (dsRNA). Furthermore, during manufacturing and storage, RNA can be degraded by exposure to heat, hydrolysis, oxidation, light, and ribonucleases. Variability may also be introduced into therapeutics by batch-to-batch manufacturing. Accordingly, analysis of manufactured mRNA is required for quality assurance.

Typical mRNA length is between about 2,000-5,000 nucleotides (0.6-1.5 MDa). Such large molecules are difficult to characterize by traditional methods of polynucleotide analysis, including methods of oligonucleotide separation and mass spectrometry. Such characterization and quantification may be essential to assessing purity of synthesis and determining pharmacokinetic and pharmacodynamic parameters of therapeutic polynucleotides. To overcome the limitations of characterizing larger polynucleotides, they are typically first digested into smaller (shorter) fragments for analysis. However, very small fragments can be difficult to accurately map, and, therefore, may be less informative, particularly with respect to primary sequence. Accordingly, there can be an optimal range of fragment sizes which may facilitate polynucleotide analysis. Yet, there are presently insufficient methods available for fragmenting larger polyribonucleotides (e.g., mRNAs) into fragments of suitable or optimal size for polynucleotide analysis.

Therefore, there exists a need for improved methods of characterizing large polyribonucleotides. More specifically, there is a need for methods of polyribonucleotide digestion that can efficiently digest polyribonucleotides into fragments having more controllable length distributions, such as those that are more suitable for polynucleotide analysis (including, for example, by liquid chromatography and/or mass spectrometry) and that are adaptable enough to be applied to polyribonucleotides (e.g., mRNAs) of variable sequence identity.

### SUMMARY

The disclosure herein is generally related to improved methods, kits, and systems for digesting and characterizing large RNA molecules. Large RNA molecules are difficult to characterize by traditional methods, including liquid chromatography and mass spectrometry. Digesting large RNA molecules into smaller fragments, particularly with sequence-specific nucleases, can allow for easier characterization and mapping of RNA fragments, but unrestricted cleavage of RNA, particularly with less selective ribonucleases (recognizing short motifs) may lead to fragments that are too small to effectively map and/or a plurality of isobaric fragments that are difficult to distinguish. The technology described herein relates to the selective formation of oligonucleotide duplexes with single stranded RNA so that nucleases that are not conventionally used to cleave single-stranded RNA, including nucleases that traditionally have been known only to cleave double-stranded DNA, may be repurposed to selectively cleave large RNA molecules in a site-specific manner into fragments of controllable/predetermined sizes. Accordingly, the precise size ranges of RNA fragments from a digestion may be tailored for polynucleotide analysis, including by liquid chromatography and/or mass spectrometry.

According to one aspect of the disclosure, provided herein is a method of digesting an RNA molecule having a known reference sequence into smaller RNA fragments. The method entails forming one or more oligonucleotide duplexes with the RNA molecule along specific portions of the reference sequence. The RNA molecule is then digested into the fragments with one or more sequence-specific nucleases that cleave the RNA molecule at a plurality of predetermined sequence-specific sites. One or more sequence-specific nucleases are duplex-dependent nucleases that only act on RNA within a duplex. Each of the one or more duplexes formed with the RNA molecule has a motif recognized by one of the one or more duplex-dependent nucleases. Embodiments of the method may include one or more of the following features.

The method may use a plurality of sequence-specific nucleases to digest the RNA molecule. The plurality of nucleases may be a plurality of duplex-dependent nucleases. A sequence-specific duplex-dependent nuclease may be a restriction endonuclease, a CAS protein, an artificial site-specific RNA endonuclease (ARSE), an enzyme comprising an RNase III domain, or a deoxyribozyme. A duplex-dependent nuclease which is a restriction endonuclease may be AvaII, AvrII, BanI, TaqI, HinfI, or HAEIII. Other sequence-specific nucleases employed may be RNase T1, RNase A, Colicin E5, or MazF.

The RNA molecule may have a length greater than about 1,000 mers. In some embodiments, the RNA fragments may be between about 6 to 1,000 mers in length, more specifically about 6 to 500 mers in length, even more specifically about 6 to 50 mers in length, or further specifically about 6 to 20 mers in length. In some embodiments, the RNA fragments may be between about 10 to 1,000 mers in length, more specifically about 10 to 500 mers in length, even more specifically about 10 to 50 mers in length, or further specifically about 10-20 mers in length. In some instances, the RNA fragments may be about 20 mers in length.

The one or more duplexes may be a plurality of duplexes. Each of the one or more duplexes may be formed with the RNA molecule and another oligonucleotide that is between about 10 and 50 mers in length. Each of the one or more duplexes may be formed by hybridizing an exogenous oligonucleotide with the RNA molecule. The one or more duplexes may be formed with DNA oligonucleotides.

At least one of the sequence-specific nucleases may be immobilized on a solid support. The immobilized nuclease may be provided in the form of an immobilized enzyme reactor (IMER) that allows flow-through digestion of the RNA molecule. The nuclease immobilized within the IMER may not be a duplex-dependent nuclease and may be used to further digest a selected fraction of the RNA fragments already digested with a duplex-dependent nuclease.

The RNA molecule may be an mRNA molecule. The plurality of predetermined sequence-specific sites may include a site within about 100 nucleotides of a proximal end of a 3′ poly(A) tail and/or a site within about 100 nucleotides of a 5′ cap.

The method may further entail separating one or more of the RNA fragments based on length using liquid chromatography. The method may further entail measuring the mass of one or more of the RNA fragments using mass spectrometry. The method may further entail mapping the RNA fragments to the reference sequence.

According to another aspect of the disclosure, provided herein is a kit for digesting an RNA molecule having a reference sequence into smaller RNA fragments. The kit includes a plurality of oligonucleotides. Each oligonucleotide is configured to hybridize to a single unique portion of the RNA molecule and has a motif that is recognized by a sequence-specific duplex-dependent nuclease that only acts on RNA within a duplex. Embodiments of the kit may include one or more of the following features.

Each of the oligonucleotides may be between about 10 to 50 mers in length. In some embodiments, each of the oligonucleotides is between about 15-25 mers in length. The plurality of oligonucleotides may include at least two motifs recognized by different sequence-specific duplex-dependent nucleases. The kit may further include one or more the sequence-specific duplex-dependent nucleases.

According to another aspect of the disclosure, provided herein is another kit for digesting an RNA molecule into smaller RNA fragments. The kit includes a plurality of sequence-specific nucleases, at least one of which is a duplex-dependent nuclease that only acts on RNA within a duplex and at least one of which is a ribonuclease that acts on single stranded RNA. The at least one duplex-dependent nuclease may be or may include a restriction endonuclease.

According to another aspect of the disclosure, provided herein is a system for mapping RNA fragments to a reference sequence. The system has a detector configured to quantify amounts of RNA oligonucleotides between about 20 and 1,000 mers in length and a processor operably connected to the detector. The processor is programmed to map detected RNA oligonucleotides to a reference sequence of an RNA molecule based at least in part on the length or mass of the detected RNA oligonucleotides. Mapping the detected RNA oligonucleotides to the reference sequence entails the processor determining the length of fragments that should be produced by digesting the RNA molecule into smaller fragments according to any embodiment of the aforementioned method. Embodiments of the system may include one or more of the following features.

The processor may be further configured to automatically identify motifs within the reference sequence for which cleavage with the sequence-specific nucleases would result in fragments between about 20 and 1,000 mers in length. The sequence-specific cleavages may be or may include one or more selective cleavages with the one or more duplex-dependent nucleases of the aforementioned method. The processor may be operably connected to one or more databases having a plurality of sequence-specific nucleases and motifs corresponding to each of the sequence-specific nucleases.

### BRIEF DESCRIPTION OF THE DRAWINGS

The technology will be more fully understood from the following detailed description taken in conjunction with the accompanying drawings, in which:

FIG. 1 is an HPLC chromatogram of a ladder of 15-100 mer oligodeoxythymidines;

FIG. 2A is a simulated HPLC chromatogram of RNA fragments generated from the digestion of a COVID-19 mRNA vaccine with TaqI restriction sites; and

FIG. 2B is a simulated HPLC chromatogram of RNA fragments generated from the digestion of the COVID-19 mRNA vaccine with select TaqI, AvaII, and BanI restriction sites.

### DETAILED DESCRIPTION

### Polynucleotide Analysis and Fragment Size

Disclosed herein are methods of digesting RNA (polyribonucleotides) which are amenable to producing RNA fragments within more controllable size ranges than standard RNA digestion methods. It will be understood that other types of oligonucleotides (e.g., DNA) may be readily substituted for the RNA to be digested in the methods, kits and systems described herein, with the selection of enzymes for digestion being dependent on the specific oligonucleotide compositions. In specific implementations, the size ranges are optimal for polynucleotide analysis. Polynucleotide analysis may be performed to determine or confirm the length, molecular weight, purity, capping status, and/or primary sequence of a sample of polynucleotide. The analysis may be used to characterize a distribution of any one or more variables where the sample is heterogeneous. Competing factors with respect to polynucleotide size can complicate polynucleotide analysis, including polynucleotide mapping, such as by liquid chromatography and mass spectrometry (including tandem mass spectrometry). Generally, larger (longer) polynucleotides are more difficult to characterize by separation methods, such as liquid chromatography. Larger polynucleotides are also generally more difficult to characterize by mass spectrometry, whereas smaller (shorter) oligonucleotides are easier to analyze. Without being limited by theory, smaller oligonucleotides are more amenable to producing intact mass measurements. Larger oligonucleotides are also more prone to salt adduction, multiple charge states, and reduced ionization and fragmentation efficiency, complicating mass spectrometry analysis. However, the characterization of shorter oligonucleotides (e.g., 2, 3, 4, 5, and 6 mer oligonucleotides) may be less informative as shorter sequences are less likely to be unique occurrences within a large oligonucleotide sequence (e.g., especially one greater than 1,000, 1,500, 2,000 mers etc.) and, therefore, may map to multiple locations within a target oligonucleotide. Also, compressing all of the primary sequence information into a small range of very short oligonucleotide fragments increases the probability of producing isobaric fragments, particularly given the limited selection of available nucleotides, which cannot be distinguished via mass spectrometry or can only be distinguished via complex and difficult analysis with tandem mass spectrometry (MS/MS).

In some embodiments, the digestion methods described herein are used to generate one or more fragments from a larger oligonucleotide that may each be uniquely mapped to the larger oligonucleotide. In some instances, the fragments are at least about 10, 15, 20, 25, 30, 35, 40, 45, or 50 mers in length. In some embodiments, the digestion methods described herein are used to generate one or more fragments from a larger oligonucleotide that are readily separable (e.g., by liquid chromatography). In some embodiments, the digestion methods described herein are used to generate one or more fragments from a larger oligonucleotide that are optimally sized for accurate mass determinations by mass spectrometry and/or tandem mass spectrometry. In some instances, the fragments are no greater than about 2,000, 1,500, 1,000, 500, 200, or 100 mers in length. In some instances, the one or more fragments may be no greater than about 90, 80, 70, 60, 50, 45, 40, 35, 30, 25, 20, 15, or 10 mers in length. In certain specific embodiments, the one or more fragments may be between about 10-100, 10-50, 10-25, 15-100, 15-50, 15-25, 20-100, 20-50, 25-100, 25-50, 30-100, 30-50, 35-100, 30-50, 40-100, or 40-50 mers in length. In various implementations, fragments which are characterized only by liquid chromatography may be longer than those to be characterized by mass spectrometry. In various embodiments, at least about 75, 80, 85, 90, 95, 96, 97, 98, or 99% of the fragments generated (by number or mass percentage) or all of the fragments generated fall within one or more of a preselected size range, including any one or more of the ranges described herein.

In various embodiments, the methods of digestion described herein are performed on a target oligonucleotide comprising ribonucleotides (a target RNA) or on a sample of analyte comprising a target RNA (e.g., with potential impurities), which may be referred to as an “RNA sample.” In some instances, the RNA sample is a synthetically manufactured RNA (e.g., mRNA), such as for therapeutic purposes. The target RNA may be a large RNA molecule having a reference RNA sequence for which it would be useful, with respect to polynucleotide analysis, to divide the target RNA molecule into smaller fragments for analysis. The target RNA molecule may be at least about 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 3,000, 3,500, 4,000, 4,500, or 5,000 mers. In certain embodiments, the target RNA molecule is at least about 1,000 mers. In certain other embodiments, the target RNA molecule is at least about 2,000 mers. Still, in certain other embodiments, the target RNA molecule is at least about 5,000 mers. In some embodiments, use of the methods of digestion, described herein, on the target RNA molecule may result in a plurality of cleavages within the target RNA molecule (e.g., at least about 2, 3, 4, 5, 6, 7, 8, 9, 10, 15, 20, 25, 30, 35, 40, 45, or 50 cleavages per target molecule). In some embodiments, the cleavages may result in a distribution of fragments having unique lengths, which may be advantageous for polynucleotide analysis. In certain embodiments, each fragment which is analyzed or mapped may have a length that is at least about 2, 3, 4, 5, 6, 7, 8, 9, 10, 15, 20, 25, 30, 35, 40, 45, 50, 60, 70, 80, 90, or 100 mers different than any other fragment. In various embodiments, the methods described herein will allow for mapping at least about 75%, 80%, 85%, 90%, 95%, 96%, 97%, 98%, 99% or 100% of a target RNA molecule (percent sequence coverage).

### Nucleases for Digesting RNA

The digestion methods described herein may be used to digest large target RNA molecules into fragments within optimal size ranges for polynucleotide analysis, which are described elsewhere herein. Digestion of RNA may be performed with one or more enzymes (nucleases) that cleave the phosphodiester bonds between ribonucleotides (e.g., 1, 2, 3, 4, 5, or more nucleases). As will be understood to those of ordinary skill in the art, some nucleases may cleave only single-stranded nucleic acids and may or may not be specific to DNA or RNA. Some nucleases may cleave only double-stranded nucleic acids (duplexes) and may or may not be specific to DNA/DNA duplexes (i.e., DNA duplexes), RNA/RNA duplexes (i.e., RNA duplexes), or DNA/RNA duplexes (i.e., heteroduplexes). As used herein, a “duplex” may refer to a region of a nucleic acid molecule in which two oligonucleotide strands are reversibly bound to each other by Watson-Crick base-paring (hydrogen bonds between complementary nucleotides within the duplex). A duplex may be formed from complete or full complementarity of base pairs along the length of the double-stranded region or by partial (e.g., substantial) complementarity (e.g., allowing for one or more mismatches which do not preclude the hybridization of the two oligonucleotide strands under relevant hybridization conditions). In some embodiments, a duplex acted on by the digestion methods described herein may be an RNA duplex where both strands are RNA. In some embodiments, the duplex may be a heteroduplex where an RNA strand, such as that of a target RNA molecule, is bound to a DNA strand. In some embodiments, the RNA molecule and/or the other oligonucleotide within a duplex may comprise modified nucleotides, including, for example, oligonucleotides which comprise both deoxyribonucleotides and ribonucleotides. The duplex may not extend the entire length of the molecule. The portion of the RNA molecule which is duplexed may be relatively small (e.g., no more than about 30%, 20%, 10%, 5% of the length of the molecule). For example, as described in detail herein, a single large RNA molecule may simultaneously form multiple duplexes with multiple smaller oligonucleotides at various positions along the length of the RNA molecule. In some embodiments, portions of the RNA molecule which are to be shielded from cleavage with ribonucleases that act on single-stranded RNA (e.g., RNase T1) are duplexed. In such embodiments, the methods of digestion described herein may be modified to forego the use of duplex-dependent nucleases such that the duplexes allow the (negative) selection of portions of the RNA molecule susceptible to cleavage with sequence-specific ribonucleases that act on single stranded RNA.

The nucleases described herein may cleave nucleic acids at sequence-specific sites. As used herein, “sequence-specific” indicates that for a given nuclease and a given target sequence the precise cleavage site(s), if any, will be determinable with certainty, under appropriate reaction conditions and assuming, for example, no secondary structures preclude cleavage. Such sequence-specific nucleases may recognize sequence “motifs” which determine precisely where a target oligonucleotide will be bound and cleaved by the nuclease. As used herein, a “DNA motif” may refer to a motif that is recognized in a DNA strand and an “RNA motif” may refer to a motif that is recognized in an RNA strand. Depending on the particular nuclease, where the nuclease has catalytic activity against each strand of a duplex, a motif in one strand of a duplex may be understood to have a complementary motif in the other strand of the duplex. It should be understood that where a nuclease has activity against both DNA and RNA an equivalent RNA motif may be readily determined from the DNA motif (i.e. uracil (U) may be readily substituted for thymine (T) where present in a motif). Accordingly, where such a nuclease is referenced, a reference to the nuclease's motif may be understood to refer to the motif of either strand, depending on the context. Similarly, it will be understood that, unless indicated otherwise, a modified oligonucleotide may be substituted for a corresponding non-modified nucleotide within a motif (as is recognized by the native enzyme) or, more generally, anywhere within an oligonucleotide acted upon by the nuclease, without substantially hindering enzymatic activity. For instance, modified NI-methyl-pseudouridine (Ψ), as is found in various mRNA vaccines, may generally be substituted for uridine without loss of activity.

Methods of digestion of RNA disclosed herein may be performed with only sequence-specific nucleases such that sequences and length of fragments produced by digestion of an RNA molecule of a known sequence from the one or more nucleases may be predicted with certainty, assuming each targeted cleavage site is in fact cleaved. Some sequence-specific nucleases may cleave oligonucleotides at a fixed position within the recognition motif. Some nucleases may cleave oligonucleotides at a fixed position (e.g., defined by a number of nucleotides) outside of a recognition motif such that, for the purposes of the instant disclosure, the nuclease may be considered sequence-specific since the exact cleavage site can be predicted from a known target sequence having the recognition motif. In some embodiments, a plurality of sequence-specific nucleases is used to digest RNA (e.g., 2, 3, 4, 5, or more sequence-specific nucleases).

Various sequence-specific nucleases may have varying degrees of selectivity relative to potential target oligonucleotides with less selective nucleases tending to produce more cuts in a target oligonucleotide than more selective nucleases. This may be particularly so with respect to a large target oligonucleotide which is generally more likely to exhibit higher sequence diversity (at least over smaller scales) relative to a smaller oligonucleotide target. Motif length (i.e., the number of nucleotides in a motif) may correlate with nuclease selectivity, wherein nucleases that recognize longer motifs are generally more selective than nucleases that recognize shorter motifs. In some embodiments, more selective nucleases are preferred in order to produce fewer targeted cuts in a target RNA molecule and, therefore, longer fragments, on average, for at least one of the nucleases used in a digestion. In some embodiments, more selective nucleases are preferred for a plurality of nucleases used in a digestion (e.g., each of the nucleases). In some embodiments, a nuclease may recognize a motif that is 3, 4, 5, 6, 7, or more nucleotides in length. Such nucleases may be used in combination with nucleases that recognize shorter motifs (e.g., 1 and/or 2 nucleotides in length). Combinations of nucleases with different degrees of selectivity may be employed, including in targeted fashions, as described elsewhere herein.

According to the methods disclosed herein, at least one of the nucleases used in a digestion is a sequence-specific “duplex-dependent nuclease of RNA” (i.e., a nuclease which only cleaves an RNA molecule when bound to a duplex formed within the RNA molecule). In some embodiments, the duplex must be a heteroduplex. In some embodiments, the duplex must be an RNA duplex. In some embodiments, the duplex may be either a heteroduplex or an RNA duplex. In some embodiments, a duplex-dependent nuclease of RNA will produce two blunt ends. In some embodiments, a duplex-dependent nuclease of RNA will produce two overhangs or “sticky ends.” In some embodiments, a plurality of sequence-specific duplex-dependent nucleases of RNA are used in a digestion (e.g., 2, 3, 4, 5, or more). In some embodiments, one or more sequence-specific duplex-dependent nucleases of RNA may be used in combination with one or more sequence-specific nucleases that are not duplex-dependent nucleases of RNA (e.g., standard ribonucleases (RNases)) to digest RNA.

Given that naturally occurring RNA is generally found in single-stranded forms (although large portions may be self-hybridized in secondary structures, such as loops and stems, via Watson-Crick base pairing), many such sequence-specific duplex-dependent nucleases of RNA are deoxyribonucleases (DNases) that are found in nature to cleave DNA duplexes, but which have been discovered to, nonetheless, exhibit sufficient catalytic activity against other types of duplexes comprising RNA (e.g., heteroduplexes). Because the sequence-specific forms of deoxyribonucleases, including some nucleases of DNA duplexes, have generally been known to show higher sequence selectivity than the most selective sequence specific forms of ribonucleases, the use of compatible sequence-specific deoxyribonucleases on target RNA, particularly duplexed RNA, may achieve higher selectivity in the digestion of RNA than the use of typical ribonucleases, allowing more targeted cuts that can more readily produce RNA fragments within desired size ranges. However, the selective formation of duplexes, as described elsewhere herein, may advantageously increase the selectivity of sequence-specific duplex-dependent nucleases of RNA, even where the nuclease is generally less selective (allowing more targeted cuts). The formation of duplexes within the target RNA prior to cleavage may be further advantageous for digestion and analysis of target RNA as the duplexes can prevent/disrupt the formation of secondary structures in sample RNA which might otherwise result in a missed cleavage by use of standard ribonucleases on single-stranded target RNA. While partial digestion with ribonucleases that act on single-stranded RNA and are therefore prone to missed cleavages of motifs within secondary structures may advantageously produce longer fragments than would be expected from complete digestion with the ribonuclease, as described, for example, in Vanhinsbergh, et al., Anal Chem. 2022 May 24; 94(20):7339-7349 (doi: 10.1021/acs.analchem.2c00765), RNA mapping can become very complex from considering the large number of putative clips that could be formed by partial digestion and repeatability of mRNA mapping experiments may be hindered. Accordingly, use of duplex-dependent nucleases of RNA may provide advantages in predictability of cleavages over use of sequence-specific ribonucleases that act on single-stranded RNA under conditions that promote missed cleavages in order to induce larger fragment size.

### Representative Sequence-Specific Duplex-Dependent Nucleases of RNA

Various nucleases, including native enzymes and engineered enzymes, are known in the art which may function as a sequence-specific duplex-dependent nuclease of RNA according to the methods described herein. In some embodiments, a sequence-specific duplex-dependent nuclease of RNA may be a restriction endonuclease (restriction enzyme). Restriction endonucleases are nucleases that cleave DNA duplexes into fragments at or near specific recognition sites within molecules known as restriction sites. All restriction endonucleases cut the sugar-phosphate backbone of both strands of a DNA double helix. Various examples of restriction endonucleases including their respective motifs are described in the REBASE™ database (available at re3data.org/repository/r3d100012171), which is a publicly available database. Restriction endonucleases are commonly classified into five types, which differ in their structure and whether they cut their DNA substrate at their recognition site, or if the recognition and cleavage sites are separate from one another. In some embodiments, the restriction endonuclease is a type II restriction endonuclease. Type II restriction endonucleases usually cleave each strand of a duplex at a specified site within the recognition motif itself. They do not use ATP or AdoMet for their activity. They usually require only Mg²⁺ as a cofactor. Some type II restriction endonucleases cut duplexes to form two blunt ends whereas others form two overhangs or “sticky ends.”

In some embodiments, the sequence-specific duplex-dependent nuclease of RNA may be a type IIP restriction endonuclease. In some embodiments, the duplex-dependent nuclease of RNA may be a member of the structural class that employs a canonical PD-(E/D)XK catalytic motif to affect cleavage (e.g., Avail, AvrII, BanI, TaqI, or HinfI). In some embodiments, the duplex-dependent nuclease of RNA may be MvaI or BanI. Type IIP restriction endonucleases form homodimers and recognize palindromic motifs that 4-8 nucleotides in length. They generally cleave within the recognition motif. In some embodiments, a duplex-dependent nuclease of RNA recognizes a palindromic motif. In some embodiments, a duplex-dependent nuclease of RNA recognizes a motif that is 4-8 nucleotides in length. Type IIP enzymes specific for 6-8 bp sequences mainly act as homodimers, composed of two identical protein chains that associate with each other in opposite orientations (e.g., EcoRI, HindIII, BamHI, NotI, PacI). Each protein subunit binds roughly one-half of the recognition sequence and cleaves one DNA strand. Since the two subunits are identical, the enzyme is symmetric, and so the overall recognition sequence, and the positions of cleavage, are also symmetric. Usually, these enzymes cleave both DNA strands at once, each catalytic site acting independently of the other. Type IIP enzymes that recognize shorter, 4-bp, sequences often act as monomers composed of a single protein chain (e.g., MspI, HinP1I, BstNI, NciI.) These have only one catalytic site, and upon binding, cleave only one strand. However, because they recognize sequences that are palindromic, they can bind in either orientation and ultimately cleave both strands, first one and then the other. The switch in enzyme orientation that takes place is usually very fast, with little accumulation of ‘nicked’ intermediate molecules cleaved in only the first strand. Other Type IIP enzymes (e.g., SfiI, NgoMIV) act as complex homotetramers—dimers of homodimers—or higher order oligomers that bind to and cleave two or more recognition sequences at once.

Depending on how close the subunits of Type IIP homodimers are to each other, the sequence recognized can be continuous (e.g., EcoRI: GAATTC), or discontinuous, with one (e.g., HinfI: GANTC), two (e.g., Cac8I: GCNNGC), three (e.g., A1wNI: CAGNNNCTG), four (e.g., PshAI: GACNNNNGTC), five (e.g, Bg1I: GCCNNNNNGGC), or more unspecified bp (N), up to nine unspecified bp (e.g., XcmI. CCANNNNNNNNNTGG). Type IIP enzymes cleave their recognition sequences at a variety of positions, depending on where the catalytic site is positioned in the protein relative to the sequence-recognition residues. Some generate 5′-overhangs (‘staggered ends’) of four bases (e.g., HindIII: A/AGCTT) or of two bases (e.g., NdeI. CA/TATG). Others generate 3′-overhangs of four bases (e.g., SacI: GAGCT/C) or two bases (e.g,. PvuI. CGAT/CG). And yet others produce blunt ends (e.g., EcoRV: GAT/ATC). Enzymes with ambiguous base pairs in their recognition sequences can generate ends with an odd number of bases, including one base (e.g., NciI. CC/SGG), three bases (e.g., TseI. G/CWGC), five bases (e.g., PspGI:/CCNGG), or more.

Most Type IIP enzymes recognize sequences that are unique, in which only one specific base pair can be present at each position (e.g., Bg1II: AGATCT). However, some recognize “degenerate” or “ambiguous” sequences in which alternative bases can be present. The most common degenerate nucleotides are Y (pyrimidine, C or T) and R (purine, A or G) (e.g., ApoI. RAATTY). Others are M (modifiable base, A or C) and K (non-modifiable base, G or T) (e.g., AccI. GTMKAC); W (weak hydrogen bonding, A or T) (e.g., BstNI: CCWGG); and S (strong hydrogen bonding, C or G) (e.g., NciI. CCSGG). The atomic structure of the enzyme's binding site determines which base pair(s) can be recognized at each position.

Murray et al., Nucleic Acids Res. 2010 December; 38(22):8257-68 (doi: 10.1093/nar/gkq702), which is herein incorporated by reference in its entirety, provides several examples of DNA endonucleases which have been found to exhibit activity against RNA in heteroduplexes. See also, Kisiala et al., Nucleic Acids Res. 2020 Jul. 9; 48(12):6954-6969 (doi: 10.1093/nar/gkaa403), which is herein incorporated by reference in its entirety. Table 1 below depicts the motifs and functionality of several DNA endonucleases which exhibit such activity. In some embodiments, one or more (e.g., 1, 2, 3, 4, 5, or 6) of the nucleases from Table 1 are employed in a method of digestion described herein as a sequence-specific duplex-dependent nuclease of RNA. In some implementations, one or more sequence-specific duplex-dependent nucleases of RNA may be selected as needed to achieve fragments within desired size ranges in order of relative activity toward RNA in such duplexes, with more active nucleases being selected before less active nucleases. For example, in some instances, some such nucleases may be selected in the relative order of TaqI, AvaII, AvrII, BanI, HinfI. In some instances, some such nucleases may be selected in the relative order of AvaII, MvaI, BanI. Other DNA endonucleases which may function as sequence-specific duplex-dependent nucleases of RNA, include, for example, MvaI (motif: CC/WGG) and BanI (motif: CC/SGG), which are both type IIP endonucleases.

TABLE 1
Exemplary sequence-specific DNA endonucleases that exhibit duplex-dependent
cleavage of RNA

| | | Experimental Findings in |
|---|---|---|
| Nuclease | Motif (5′-3′) | Heteroduplexes |
| AvaII | G/GWCC, W = A or T | cleaves DNA and RNA strands |
| AvrII | C/CTAGG | cleaves DNA and RNA strands |
| BanI | G/GYRCC, Y = C or T; R = A or G | cleaves DNA and RNA strands |
| TagI | T/CGA | cleaves DNA and RNA strands |
| HinfI | G/ANTC, N = A, C, G or T | cleaves RNA strand |
| HAEIII | GG/CC | cleaves RNA strand |

In some embodiments, a sequence-specific duplex-dependent nuclease of RNA may be a CRISPR-associated system (Cas) protein. CRISPR (clustered regularly interspaced short palindromic repeats) is a family of DNA sequences found in the genomes of prokaryotic organisms such as bacteria and archaea, that provide immunity against plasmids and bacteriophage by using foreign DNA stored as CRISPR spacer sequences together with Cas nucleases to stop infection. More recently, CRISPR-CAS systems have been effectively repurposed for gene editing. Like restriction endonucleases, most CRISPR-CAS systems catalyze the cleavage of double-stranded DNA, or occasionally single stranded DNA. CRISPR technology is well known in the art. Briefly, CAS proteins such as the more commonly employed CAS9 protein for gene editing, usually require a CRISPR RNA (crRNA) that recognizes a target sequence via Watson-Crick binding and a trans-activating RNA (tracrRNA) that forms a duplex region with a portion of the crRNA allowing complexing with the Cas protein. The crRNA and tracrRNA may be joined into a single-guide RNA (sgRNA), generally having a hairpin loop. Thus, the crRNA/sgRNA provide sequence specificity to the Cas nuclease. Many Cas proteins also require recognition of a protospacer adjacent motif (PAM) sequence adjacent to the target sequence. However, this is ultimately not too limiting, as it is typically a very short and nonspecific sequence that occurs frequently at many places throughout a genome (e.g., the SpCas9 PAM sequence is 5′-NGG-3′ and in the human genome occurs approximately every 8 to 12 base pairs).

More recently, it has been discovered that the CAS13 protein targets single-stranded RNA rather than DNA for cleavage and may be programmed to be sequence specific. CAS13 is described in further detail in Wessels, et al., Nat Biotechnol. 2020 June; 38(6):722-727 (doi: 10.1038/s41587-020-0456-9); Abudayyeh, et al., Science. 2016 Aug. 5; 353 (6299):aaf5573 (doi: 10.1126/science.aaf5573); East-Seletsky, et al., Nature. 2016 Oct. 13; 538(7624):270-273 (doi: 10.1038/nature19802); Mol Cell. 2015 Nov. 5; 60(3):385-97 (doi: 10.1016/j.molce1.2015.10.008), each of which is herein incorporated by reference in its entirety. It has also been shown that the S. pyogenes Cas9 (SpyCas9) can be supplied with a short DNA oligo containing the PAM sequence (a PAMmer) to induce single-stranded RNA (ssRNA) binding and cutting.

Furthermore, it has more recently been shown that Cas9 enzymes from both subtypes II-A and II-C can recognize and cleave single-stranded RNA (ssRNA) by an RNA-guided mechanism that is independent of a PAM sequence in the target RNA. RNA-guided RNA cleavage is programmable and site-specific. RNA cleavage by Cas9 is described in further detail in Strutt et al., Elife. 2018 Jan. 5; 7:e32724 (doi: 10.7554/eLife.32724), which is herein incorporated by reference in its entirety. In some embodiments, the sequence-specific duplex-dependent nuclease of RNA may be a Cas protein (e.g., a Cas13 or Cas9 protein). In some embodiments, the nuclease is a subtype II-A or II-C Cas9 protein. In some embodiments, the nuclease is S. aureus Cas9 (SauCas9) or C. jejuni Cas9 (CjeCas9) protein. In various embodiments employing a CAS protein, a crRNA or sgRNA may effectively function as an exogenous nucleotide, as described elsewhere herein, which forms a duplex for promoting sequence-specific duplex-dependent cleavage of RNA.

In some embodiments, a sequence-specific duplex-dependent nuclease of RNA may be an artificial nuclease. For example, in some embodiments, one or more sequence-specific duplex-dependent nucleases of RNA may be an artificial site-specific RNA endonuclease (ASRE) as described in Choudhury et al., Nat Commun. 2012; 3:1147 (doi: 10.1038/ncomms2154), which is herein incorporated by reference in its entirety. Briefly, an ASRE may comprise an RNA binding PUF domain, which can be engineered to specifically bind any 8 nucleotide RNA motif, linked to a PIN domain for cleaving RNA. Other artificial nucleases, including nucleases using PUF domains to recognize specific RNA motifs, may be used in the methods described herein.

In some embodiments, a sequence-specific duplex-dependent nuclease of RNA may be an enzyme within the RNase III family, characterized by a RNase III catalytic domain. These enzymes recognize and cleave double-stranded RNA (dsRNA) at specific sites. They are ubiquitous enzymes in cells that play a major role in pathways such as RNA precursor synthesis, RNA silencing, and the pnp autoregulatory mechanism. The enzyme may be a class 1, class 2, class 3, or class 4 RNase III. In some embodiments, the RNase III enzyme may be Dicer. Dicer cleaves dsRNA and pre-microRNA (pre-miRNA) in vivo into short double-stranded RNA fragments called small interfering RNA and microRNA, respectively. These fragments are approximately 20-25 base pairs long with a two-base overhang on the 3′-end. Dicer facilitates the activation of the RNA-induced silencing complex (RISC), which is essential for RNA interference. Human dicer comprises two RNase III domains, two double stranded RNA binding domains (DUF283 and dsRBD), a helicase domain, and a PAZ (Piwi/Argonaute/Zwille). Current research suggests the PAZ domain is capable of binding the 2 nucleotide 3′ overhang of dsRNA while the RNase III catalytic domains form a pseudo-dimer around the dsRNA to initiate cleavage of the strands. Dicer may work cooperatively with other regulatory proteins in order to effectively position the RNase III domains and thus control the specificity of the sRNA products. In some implementations, additional regulatory proteins are used in combination with Dicer. Dicer is described in additional detail in Paturi et al., Front Mol Biosci. 2021 May 7; 8:643657 (doi: 10.3389/fmolb.2021.643657), which is herein incorporated by reference in its entirety. In some embodiments, the RNase III enzyme may be Drosha. Drosha is the primary nuclease that executes the initiation step of miRNA processing in the nucleus. It works closely in vivo with DGCR8 and in correlation with Dicer. In some implementations, additional regulatory proteins are used in combination with Drosha. Drosha and Dicer are described in more detail in Leitao, et al., Noncoding RNA. 2022 Jan. 18; 8(1):10 (doi: 10.3390/ncrna8010010), which is herein incorporated by reference in its entirety.

Other sequence-specific nucleases of dsRNA may be able to be engineered from RNase III type ribonucleotides, such as Dicer and Drosha. Glow et al., Nucleic Acids Res. 2015 Mar. 11; 43(5):2864-73 (doi: 10.1093/nar/gkv009), which is herein incorporated by reference in its entirety, describes how BsMiniIII is able to cleave long dsRNA over short time frames in a sequence-specific manner with different preferences for specific motifs (including AC/UC, and preferentially AC/CU or AG/GU) and non-specific cleavage occurring over longer time frames, proposing the enzyme as a prototype for engineering sequence-specific ribonucleases. In some implementations, a nuclease may be considered sequence-specific if it only performs sequence-specific reactions over the time frame of the digestion reaction.

In some embodiments, one of the one or more sequence-specific nucleases used to digest RNA may be a deoxyribozyme (also known as a DNA enzyme, DNAzyme, or catalytic DNA). Deoxyribozymes are DNA oligonucleotides capable of performing specific, usually catalytic, chemical reactions. The most abundant class of deoxyribozymes are ribonucleases, which catalyze the cleavage of a ribonucleotide phosphodiester bond through a transesterification reaction, forming a 2′3′-cyclic phosphate terminus and a 5′-hydroxyl terminus. Most but not all of these deoxyribozymes require a divalent metal ion cofactor such as Mg²⁺ to catalyze the cleavage. While originally discovered deoxyribozymes generally recognized R/Y and A/G motifs, where R denotes a purine (A or G) and Y denotes a pyrimidine (U or C), the array of variants that have been discovered allow for the cleavage of most dinucleotide sequences N/N in vitro with reasonable rate.

The catalytic or random enzyme region of the deoxyribozyme oligonucleotide may be flanked on either or both side by binding arms that target and bind to RNA oligonucleotide targets via Watson-Crick binding. Some deoxyribozymes may preferentially employ several pairs of unmatched nucleotides near the cleavage site. The length of the binding arms may modulate binding affinity for the target RNA, with longer binding arms resulting in higher affinity. In some embodiments, long binding arms may be preferred to promote higher binding affinity and/or increased target specificity. Molar excesses of deoxyribozymes may be used to drive complete digestion under single turnover conditions. While the target motifs of deoxyribozymes are generally short (e.g., two bp), the use of binding arms comprising complementary nucleotides to the target RNA sequence may effectively increase the sequence specificity of a deoxyribozyme. To the extent that a particular deoxyribozyme will not catalyze an available target motif absent hybridization of the binding arm(s) (i.e. recognition of the a longer sequence motif by the deoxyribozyme), the deoxyribozyme may be considered a sequence-specific duplex-dependent nuclease of RNA. Effectively, the catalytic portion of the deoxyribozyme acts as the nuclease and the binding arm(s) act as the duplex-forming oligonucleotide which promotes more selective sequence-specific binding of the catalytic portion of the deoxyribozyme. Deoxyribozymes are described in more detail in Silverman, Nucleic Acids Res. 2005 Nov. 11; 33(19):6151-63 (doi: 10.1093/nar/gki930), which is herein incorporated by reference in its entirety. Similar to deoxyribozymes, peptide nucleic acid based nuclease systems (PNAzymes) may be employed in digestion of RNA as sequence-specific, or more specifically, sequence-specific duplex-dependent nucleases of RNA. PNAzymes are described in more detail in Murtola et al., J Am Chem Soc. 2010 Jul. 7; 132(26):8984-90 (doi: 10.1021/ja1008739); and Luige et al., Molecules. 2019 Feb. 14; 24(4):672 (doi: 10.3390/molecules24040672). Similarly, an aptazyme (a ribozyme fused to an aptamer), as described, for example, in Peng, et al., RSC Chem Biol. 2021 Jul. 2; 2(5):1370-1383 (doi: 10.1039/d0cb00207k), which is herein incorporated by reference in its entirety, may be considered a duplex-dependent nuclease of RNA to the extent it is engineered to only cleave an available sequence-specific motif upon recognition of a specific motif by an aptamer. To the extent any types of these enzymes are not duplex-dependent, they may be used as additional sequence-specific ribonucleases, as discussed below.

### Representative Nucleases for Additional RNA Digestion

In various implementations, one or more sequence-specific nucleases of RNA which are not duplex-dependent nucleases of RNA are used in combination with one or more sequence-specific duplex-dependent nucleases of RNA to digest target RNA. For example, 1, 2, 3, 4, 5, or more sequence-specific ribonucleases may be used to digest RNA according to the methods described herein. These sequence-specific ribonucleases may cleave single-stranded RNA. A variety of sequence-specific ribonucleases are well-known in the art, including, but not necessarily limited to, those described elsewhere herein. For example, various nucleases are described in detail in Yang, Q Rev Biophys. 2011 February; 44(1):1-93 (doi: 10.1017/S0033583510000181), which is herein incorporated by reference in its entirety, including some which may function as sequence-specific ribonucleases. In some embodiments, a sequence-specific ribonuclease may be a ribozyme as described, for example, in Peng, et al., RSC Chem Biol. 2021 Jul. 2; 2(5):1370-1383 (doi: 10.1039/d0cb00207k), which is herein incorporated by reference in its entirety. In some embodiments, a sequence-specific ribonuclease may be a nuclease described in Jiang et al., Anal Chem. 2019 Jul. 2; 91(13):8500-8506 (doi: 10.1021/acs.analchem.9b01664), which is herein incorporated by reference, and which describes digestion, characterization, and mapping of RNA with such ribonucleases.

In some embodiments, the digestion methods disclosed herein uses RNase T1 as a sequence-specific ribonuclease. RNase T1 is an endoribonuclease that specifically degrades single-stranded RNA after G residues. It cleaves the phosphodiester bond between the 3′-guanylic residue and the 5′-OH residue of adjacent nucleotides with the formation of corresponding intermediate 2′, 3′-cyclic phosphate. The reaction products are 3′-GMP and oligonucleotides with a terminal 3′-GMP. RNase T1 does not require metal ions for activity.

In some embodiments, the digestion methods disclosed herein uses RNase A as a sequence-specific ribonuclease. RNase A is an endoribonuclease that specifically degrades single-stranded RNA after pyrimidine residues (C or U). It efficiently hydrolyzes RNA by cleaving the phosphodiester bond between the 3′-phosphate group of the pyrimidine nucleotide and the 5′-ribose of its adjacent nucleotide 1, 2, 3. The intermediate 2′-,3′-cyclic phosphodiester that is generated is then further hydrolyzed to a 3′-monophosphate group.

In some embodiments, the digestion methods disclosed herein uses a Colicin as a sequence-specific ribonuclease (e.g., Colicin E5). Colicins are types of bacteriocin produced by and toxic to some strains of Escherichia coli. Colicins are released into the environment to reduce competition from other bacterial strains and bind to outer membrane receptors, using them to translocate to the cytoplasm or cytoplasmic membrane, where they exert cytotoxic effects, some of which include RNase activity. RNase-type colicins inhibit protein synthesis of sensitive cells by cleaving a specific site near the 3′ end of 16S rRNA. Colicin E5 is a known tRNase, specifically, that inhibits protein synthesis by specifically cleaving tRNATyr, tRNAHis, tRNAAsn and tRNAAsp of sensitive E. coli cells. Colicin E5 cleaves these tRNAs between the 34th queuosine (Q) and 35th uridine (U) that correspond to the first and second letters of the anticodon triplets, yielding a 2′,3′-cyclic phosphate and a 5′-OH terminus. Q is a nucleoside with a unique base, queuine, which is a highly modified guanine (G) base widely found at the aforementioned position in the above four tRNA species in prokaryotes and eukaryotes. However, Colicin E5 has been shown to exhibit RNase activity against G/U motifs as well as Q/U motifs. Colicin E5 is described in further detail in Ogawa et al., Nucleic Acids Res. 2006; 34(21):6065-73 (doi: 10.1093/nar/gk1629), which is herein incorporated by reference in its entirety.

In some embodiments, the digestion methods disclosed herein uses a MazF as a sequence-specific ribonuclease (e.g., E. Coli. Maz F or M tuberculosis MazF). MazF is a bacterial toxin that is part of MazE-MazF toxin-antitoxin system. MazF in E. Coli. is an N/ACA-specific endoribonuclease that functions independent of ribosomes and RNA codon context. The 2′—OH group in the N residue of the N/ACA cleavage motif is generally required for MazF cleavage. MazF is described in more detail in Zhang, et al., J Biol Chem. 2005 Feb 4; 280(5):3143-50 (doi: 10.1074/jbc.M411811200), which is herein incorporated by reference in its entirety. Other orthologues of MazF may recognize different 3-, 5-, or 7-residue motifs. For example, the MazF-mt6 orthologue from M. tuberculosis recognizes and cleaves a UU/CCU motif, as described, for example, in Schifano, et al., Proc Natl Acad Sci USA. 2013 May 21; 110(21):8501-6 (doi: 10.1073/pnas.1222031110), which is herein incorporated by reference in its entirety. MazF enzymes are generally expensive and their recognition motifs are relatively infrequent in mRNA, such that use of MazF enzymes alone may not provide sufficient cleavage of large RNA molecules to achieve fragments within optimal size ranges for polynucleotide analysis.

Exemplary sequence-specific ribonucleases and their motifs are depicted in Table 2 below. In various implementations, 1, 2, 3, or 4 of the ribonucleases listed in Table 2 are employed to digest target RNA in combination with one or more sequence-specific duplex-dependent nucleases of RNA.

TABLE 2
Exemplary sequence-specific Ribonucleases

| Ribonuclease | Motif (5′-3′) |
|---|---|
| RNase T1 | G/ |
| Colicin E5 | G/U |
| MazF (E. Coli) | N/ACA |
| MazF-mt6 | UU/CCU |

In some embodiments, non-specific 3′ and/or 5′ exonucleases may be used in combination with sequence-specific duplex-dependent nucleases of RNA, and optionally in combination with sequence-specific ribonucleases that are not duplex-dependent, as described elsewhere herein. Exonucleases are enzymes that work by cleaving nucleotides one at a time from the end of a polynucleotide chain by hydrolyzing the phosphodiester bonds at either the 3′ or the 5′ end. By using non-specific exonucleases, fragments from prior digestions may be further digested to generate ladders of the partially digested fragment. For example, isolated fragments from an initial digestion may be subjected to different degrees of degradation by one or more exonucleases (e.g., by longer reaction times with the exonuclease(s)). The differentially degraded fragments may be characterized, e.g., by mass spectrometry, as described elsewhere herein. The molecular weights of the differentially degraded fragments making up the ladder may be used to elucidate the sequence of the original fragment.

### Methods for Digesting RNA

Methods of using sequence-specific duplex-dependent nucleases of RNA to digest single-stranded target RNA comprises forming one or more duplexes with the target RNA and one or more other oligonucleotides. In some implementations, one or more candidate RNA motifs is identified within a reference sequence of the target RNA for each of one or more sequence-specific duplex-dependent nucleases of RNA. The candidate RNA motifs may be selected for inducing cleavage based on the expected fragment sizes that would result, to produce fragments within a desired size range or size distribution. Accordingly, the selective formation of duplexes with target RNA provides a mechanism for selectively avoiding cleavage of certain available cleavage sites within the target RNA that would otherwise be cleaved by the sequence-specific duplex-dependent nuclease of RNA, allowing further precision over the control of digestion fragment length. In some embodiments, only one candidate RNA motif for a particular sequence-specific duplex-dependent nuclease of RNA may be selected for inducing cleavage. In some embodiments, a plurality of candidate RNA motifs for a particular sequence-specific duplex-dependent nuclease of RNA may be selected for inducing cleavage (e.g., 1, 2, 3, 4, 5, 6, 7, 8, 9, 10, 15, 20, 25, 30, 40, 50 or more). In some embodiments, each available candidate RNA motif for a particular sequence-specific duplex-dependent nuclease of RNA may be selected for inducing cleavage.

For each selected RNA motif of a sequence-specific duplex-dependent nuclease of RNA, a duplex may be formed with the target RNA which encompasses the selected RNA motif. The duplex may comprise an exogenous oligonucleotide which is capable of Watson-Crick binding to the target RNA to form a sufficiently stable duplex that is in turn able to bind the particular sequence-specific duplex-dependent nuclease of RNA and promote the cleavage of the selected site. The exogenous oligonucleotide may be a DNA molecule. The exogenous oligonucleotide may be an RNA molecule. The exogenous oligonucleotide may comprise deoxyribonucleotides, ribonucleotides, and/or modified nucleotides. The exogenous oligonucleotide may have a length sufficient to form a stable enough duplex to allow for digestion under the particular digestion conditions, as will be understood by those of ordinary skill in the art. The exogenous oligonucleotide may have a length sufficient to allow binding of the particular sequence-specific duplex-dependent nuclease of RNA to the duplex in a manner sufficiently stable to promote cleavage, as will be understood by those of ordinary skill in the art. The exogenous oligonucleotide may have a length sufficient to provide enough sequence selectivity to hybridize with the selected RNA motif and not any non-selected RNA motifs. The exogenous oligonucleotide used to form a duplex with a given selected RNA motif may accordingly have a length of sequence which is sufficiently complementary to a unique region of the RNA target sequence that comprises the selected RNA motif or which is at least not sufficiently complementary to regions of the RNA target sequence which comprise non-selected RNA motifs. In some embodiments, each exogenous oligonucleotide is at least about 10, 15, 20, 25, or 30 nucleotides in length. The exogenous oligonucleotide may comprise complementary nucleotides to each nucleotide within the selected RNA motif. The entire sequence of the exogenous oligonucleotide may be fully complementary to the target RNA. In some embodiments, an individual duplex is formed for each selected RNA motif (i.e., a region of single-stranded target RNA is expected to divide the duplexes formed for each selected RNA motif).

In some embodiments, a single duplex encompasses two or more adjacent selected RNA motifs, regardless of whether the adjacent selected RNA motifs are targeted by the same or different duplex-dependent nucleases of RNA (i.e., a single exogenous oligonucleotide forms a duplex with a region of the RNA target sequence encompassing the two or more adjacent RNA motifs). In some embodiments, the one or more oligonucleotides used to form one or more duplexes with the target RNA are of lengths short enough that after digestion the exogenous oligonucleotides will not interfere with the polynucleotide analysis of the digested RNA. Accordingly, in some embodiments the lengths of the one or more oligonucleotides are selected such that after digestion the oligonucleotides will be shorter than the shortest length of any target RNA fragment to be analyzed or mapped. In some embodiments, the oligonucleotide fragments may be less than or no more than about 50, 45, 40, 35, 30, 25, 20, 15, or 10 nucleotides in length. In some embodiments, the exogenous nucleotides are about 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-30, 30-50, 30-45, 30-40, or 30-35 nucleotides in length.

In some embodiments, the method of digestion comprises using a plurality of sequence-specific duplex-dependent nucleases of RNA to digest a target RNA (e.g., at least 2, 3, 4, or 5). In some embodiments, the method of digestion comprises using one or more nucleases that are not duplex-dependent nucleases of RNA in combination with the one or more duplex-dependent nucleases of RNA. For example, the method of digestion may comprise using sequence-specific ribonucleases to make additional cleavages. In some embodiments, sequence-specific nucleases that are not duplex-dependent nucleases of RNA are used on selected fragments of the target RNA (e.g., on fraction collected separations).

Methods for enzymatic digestion of oligonucleotides in vitro, including in sample preparation for polynucleotide analysis such as by liquid chromatography and/or mass spectrometry, are well known in the art. In brief, for any given digestion reaction, a reaction mixture may be prepared comprising the sample, the nuclease, and any suitable reaction buffer for driving the enzymatic reaction (e.g., including metal ions for catalysis, such as Mg²⁺). The digestion reaction may be carried out for a predetermined amount of time prior to quenching the reaction, initiating a sequential reaction, or beginning polynucleotide analysis procedures (e.g., separation via liquid chromatography). The digestion reaction may be temperature controlled. A predetermined elevated temperature may be used to promote the digestion reaction and may be applied during a predetermined reaction time. The modulation of reaction time, reaction temperature, reaction pH, or enzyme amount (e.g., concentration) may be used to control nuclease reaction kinetics and drive complete digestion or partial digestion, as is desired. In some implementations, reaction time may be controlled by quenching an enzymatic reaction by any suitable way known in the art (e.g., temperature or pH change).

In digestion reactions involving sequence-specific duplex-dependent nucleases of RNA, exogenous oligonucleotides may be introduced into the reaction mixture for forming one or more duplex substrates to be acted upon by one or more duplex-dependent nucleases of RNA. The exogenous oligonucleotides may be introduced independently, with the duplex-dependent nucleases of RNA, with the RNA sample, with other reaction buffer components, or as combination thereof. The amount of exogenous oligonucleotides may be used to control reaction kinetics of sequence-specific duplex-dependent cleavage reactions. For example, molar excesses of exogenous oligonucleotides may be used, in combination with suitable reaction time, reaction temperature, and nuclease amounts, to ensure complete digestion of selected RNA motifs. Sub-molar amounts of exogenous oligonucleotides may be used to drive partial digestion of selected RNA motifs as desired and as is known in the art. In some embodiments, equimolar amounts of target RNA and oligonucleotides are employed.

Reaction mixtures may be incubated or mixed (e.g., via flow) during digestion reactions. Suitable conditions for annealing oligonucleotides are known in the art. In some implementations, the exogenous oligonucleotides are annealed to the sample oligonucleotides (e.g., target RNA) by creating a reaction mixture with both and then heating the reaction mixture to an elevated temperature (e.g., 95° C.) then allowing the reaction mixture to cool. The annealing process may disrupt secondary structures, as described elsewhere herein. In some instances, the nuclease may be added to the reaction mixture after annealing the exogenous oligonucleotides in order to avoid denaturing the nuclease.

In some embodiments, exogenous oligonucleotides are added to sample RNA by in vitro reverse transcription. Methods for reverse transcription are well known in the art. Single strand cDNA may be synthesized directly onto sample RNA via a single round of reverse transcription to create one or more heteroduplexes for directing cleavage by duplex-dependent nucleases of RNA as described elsewhere herein. Accordingly, reaction mixtures may comprise the sample RNA, primers (3′ primers), dNTPs, and a reverse transcriptase. In some embodiments, primers may be selected to ensure reverse transcription of all selected RNA cleavage sites (e.g., a 3′ primer complementary to a portion of the target RNA sequence positioned at the 3′ end of the desired duplex). The nuclease may be added to the reaction mixture after reverse transcription is complete.

In various embodiments, one or more of the nucleases used in a digestion described herein is immobilized onto a solid, insoluble support for performing enzymatic reactions. Various suitable supports are known in the art and include, for example, beads (which may optionally be packed into a column or which may be separated from reaction solutions by processes such as centrifugation), other particles, and membranes. In some instances, nucleases may be immobilized on magnetic beads or particles which can be magnetically isolated from a reaction solution. Immobilization of nucleases on solid substrates may facilitate the control of enzymatic reactions by removing the nucleases from reaction mixtures comprising nucleotide substrates. Immobilization may also prevent the build-up of nucleases on subsequent analytical equipment (e.g., liquid chromatography columns) from processed reaction mixtures, which could ultimately lead to undesired digestion of samples during analysis. In some embodiments, one or more nucleases are employed within immobilized-enzyme reactors (IMERs). IMERs are flow-through devices containing enzymes that are physically confined or localized with retention of their catalytic activities. IMERs can be used repeatedly and continuously and have been applied for (bio)polymer degradation, proteomics, biomarker discovery, inhibitor screening, and detection. On-line integration of IMERs with analytical instrumentation, such as high-performance liquid chromatography (HPLC) systems, reduces the time needed for multi-step workflows, reduces the need for sample handling, and enables automation. Where multiple nucleases are employed, one or more of the nucleases may be immobilized. In some instances, two or more nucleases are immobilized on the same support. In some instances, two or more nucleases are immobilized on two or more different supports. In some instances, some nucleases are immobilized and others are not (are employed in solution).

Where a plurality of nucleases is used to digest an RNA sample, the digestion reactions for any two nucleases may be performed simultaneously (in parallel) or sequentially. In some embodiments, sequential digestions may be performed before and after separation of a digested sample. For example, oligonucleotide fragments (e.g., larger fragments) may be fraction collected after separation by liquid chromatography and subjected to additional digestion reactions. In some embodiments, the subsequent digestion may be performed with a sequence-specific ribonuclease, as is described elsewhere herein, (e.g., RNase T1) which may be preferentially avoided for the first round of pre-separation digestions due to the ribonuclease's relatively low sequence specificity and the large number of cuts and small fragments it might produce on the larger undigested target RNA. Use of such ribonucleases on smaller fragments will generally produce less small fragments than on the larger undigested sample. Furthermore, the production of smaller fragments within an isolated portion of the larger RNA reference sequence is less likely to complicate mapping than the existence of smaller fragments obtained from across the entire target RNA reference sequence. In some instances, such single-stranded ribonuclease digestions may be easier to perform on-line with the fractionated separation output since no exogenous oligonucleotides are required to be introduced to complete the digestion. Such reactions can be injected into an IMER comprising the immobilized nuclease for performing the subsequent digestion reaction.

### Methods for Characterizing Digested RNA

The methods described herein may comprise performing a separation of digested polyribonucleotide fragments by length. In some embodiments, the separation is performed by chromatography. Chromatographic methods for separating oligonucleotides are well known in the art. The chromatography may be liquid chromatography. The chromatography may be reversed phase chromatography. In some embodiments, the chromatography is ion pairing chromatography, in which ion pairing reagents are mixed with the analyte prior to separation. In some implementations, a salt gradient may be applied. In some implementations, an anion exchange column may be used. In some embodiments, the chromatography is ultra high performance liquid chromatography (UHPLC). In various implementations, the liquid chromatography may be 2D-LC. In some implementations, ultraviolet detection of fragments separated by liquid chromatography (LC-UV) may be sufficient for RNA mapping. Other suitable detection methods for detecting fragments separated by LC may also be used as is known in the art. Various eluting fractions (e.g., peaks) may be stored on-line (e.g., in system loops) or off-line for later processing. On-line processing of samples (before and/or after a first round of liquid chromatography) may be performed in-line with one or more IMERs comprising one or more of the nucleases described herein for performing a digestion.

The methods described herein may comprise performing mass spectrometry on digested polynucleotide fragments. Methods for analyzing polyribonucleotide fragments by mass spectrometry are well known in the art. In some embodiments, the mass spectrometry may comprise tandem mass spectrometry (MS/MS). Methods for performing mass spectrometry may comprise charge reduction and/or data deconvolution, which are well known in the art.

The method describe herein may comprise mapping of one or more or all of digested ribonucleotide fragments to a target RNA molecule. Methods for RNA mapping are well known in the art. See, e.g., Vanhinsbergh, et al., Anal Chem. 2022 May 24; 94(20):7339-7349 (doi: 10.1021/acs.analchem.2c00765), which is herein incorporated by reference in its entirety. Suitable methods for characterizing nucleic acids, including by liquid chromatography and mass spectrometry, are described in Santos et al., J Sep Sci. 2021 January; 44(1):340-372 (doi: 10.1002/jssc.202000833); Klont et al., Drug Discov Today Technol. 2021 December; 40:64-68 (doi: 10.1016/j.ddtec.2021.10.004), each of which is herein incorporated by reference in its entirety. In some implementations, LC-MS or LC-MS/MS may be used to determine mass information prior to RNA mapping. Tandem MS/MS analysis may be used to distinguish isobaric fragments. Liquid handling systems (e.g., robotic automated liquid handling systems) as are well known in the art may be used to facilitate any one or more steps involved in the digestion or characterization processes.

Digestion and Analysis of mRNA

As mentioned elsewhere herein, the digestion methods described herein may be used on mRNA molecules. mRNA molecules generally comprise a poly(A) tail at their 3′ end and a 5′ cap at their 5′ end. The poly(A) tail and 5′ cap are generally separated from an internal coding sequence (CDS) of the mRNA molecule, which is translated into an amino acid sequence, by a 3′ untranslated region (3′ UTR) and 5′ untranslated region (5′ UTR), respectively. The poly(A) tail consists of multiple adenosine monophosphates forming a stretch of sequence of variable length, which is important for the nuclear export, translation and stability of the mRNA. The length of the poly(A) tail is heterogeneous (e.g., between about 60-120 mers) and can be difficult to control in the manufacture of mRNAs. The length or distribution of lengths of the polyA tail in an mRNA sample may be important to confirm, however the heterogeneity makes mass spectrometry analysis of intact mRNA difficult. The 5′ cap is a specially altered nucleotide which functions to regulate nuclear export, prevent exonuclease degradation, promote the initiation of translation, and promote 5′ proximal intron excision. Various 5′ cap structures exist in nature. In eukaryotes, the 5′ cap consists of a guanine nucleotide methylated on the 7 position and connected to mRNA via an unusual 5′ to 5′ triphosphate linkage (i.e., a 7-methylguanylate cap). In the manufacture of mRNAs it can be important to confirm the homogeneity and efficiency of 5′ capping. Analysis of 5′ capping is described in further detail in U.S. Pub. No. 2021/0108252 to Beverly, published Apr. 15, 2021.

The analysis of 5′ capping, poly(A) tails, and the internal mRNA sequence may each complicate the analysis of the others. For example, the heterogeneous masses/lengths of the 5′ or 3′ end within a sample may convolute the analysis of internal fragment lengths. Accordingly, it may be beneficial to analyze each separately or to at least remove the analysis of one from the analysis of the others. Methods of digestion described herein may comprise performing a targeted cleavage of the 5′ end and/or the 3′ end from an mRNA molecule prior to an analysis. In some embodiments, the internal mRNA sequence may be further digested and analyzed after removing the 5′ end and/or the 3′ end. In some embodiments, the 5′ end and/or the 3′ end may be analyzed after removing the remainder of the mRNA molecule. In various implementations, the remainder of the molecule will be sufficiently large that if left further undigested before separation it should exhibit a distinct retention behavior such that it does not interfere with the analysis of the 5′ end and/or 3′ end. Accordingly, in some embodiments, an mRNA molecule is digested into two or three clips prior to analysis. In some instances, the 5′ end and/or the 3′ end may be cleaved from the remainder of the mRNA molecule at a target cleavage site that is between 0-10, 0-20, 0-30, 0-40, 0-50, 0-60, 0-70, 0-80, 0-90, 0-100, 0-150, 50-100, 50-150, or 100-150 nucleotides away from the proximal end of the 5′ cap or the poly(A) tail.

Similarly, in some implementations, only a selected portion or segment of an RNA molecule may be desired for polynucleotide analysis. Accordingly, one or two selective cleavages may be made in the RNA molecule according to the methods described herein to isolate that segment for analysis. Additional digestions may be subsequently performed on the selected segment as described elsewhere herein. The non-selected portions of the RNA molecule may be disregarded to simplify the analysis of the selected segment.

### Kits and Systems for RNA Digestion/Analysis

Disclosed herein are kits and systems for performing the methods described elsewhere herein. A kit may generally comprise any two or more components required to perform a method described herein. In some embodiments, a kit comprises one or more nucleases for performing a digestion described herein, including any of the nucleases described herein. In some embodiments, a kit comprises a plurality of nucleases for performing a digestion described herein (e.g., 2, 3, 4, 5 or more nucleases). The kit may comprise at least one sequence-specific duplex-dependent nuclease of RNA. The kit may comprise a plurality of sequence-specific duplex-dependent nucleases of RNA (e.g., 2, 3, 4, 5, or more). The kit may comprise at least one sequence-specific ribonuclease which is not a duplex-dependent nuclease of RNA. The kit may comprise a plurality of sequence-specific ribonucleases which are not duplex-dependent nucleases of RNA. In some instances, the kit may comprise at least one sequence-specific duplex-dependent nuclease of RNA and at least one sequence-specific ribonuclease which is not duplex-dependent.

In some embodiments, a kit may comprise one or more solid substrates on which one or more nucleases described herein may be immobilized. In some instances, one or more of the solid substrates may be provided pre-loaded (i.e., with one or more nucleases already immobilized thereon). In some instances, one or more of the solid substrates may be provided unloaded. The solid substrates may be provided in combination with one or more nucleases for immobilizing onto the substrates. The kit may include one or more reagents for performing the immobilization chemistry. The kit may include reagents for removing enzymes from a solid support.

In some embodiments, a kit may comprise one or more reagents for performing a digestion described herein. For example, the kit may comprise suitable reaction buffers (e.g., including necessary metal ions) for carrying out a enzymatic reaction and/or for quenching an enzymatic reaction (e.g., by inducing a change in pH).

In some embodiments, a kit may comprise a one or more exogenous oligonucleotides for performing one or more of the sequence-specific duplex-dependent cleavages of RNA described herein. The one or more exogenous oligonucleotides may be configured for the digestion of an RNA molecule having a particular primary sequence. The oligonucleotides may be provided in combination with one or more nucleases recognizing one or more specific motifs within the one or more oligonucleotides. In some embodiments, a kit may comprise one or more components for reverse transcribing cDNA from an RNA sample, such as primers (e.g., 3′ primers), dNTPs, and/or a reverse transcriptase.

In some embodiments, the kit may comprise one or more components for performing polynucleotide analysis on an RNA molecule digested according to the methods described herein. For example, the kit may comprise a polynucleotide ladder or standards for performing the analysis. In some embodiments, the kit may comprise a column suitable for separating ribonucleotides within the digested size ranges by HPLC.

In some embodiments, one or more of the components for performing the digestion described herein, including, for example, the kits described herein, are provided as part of a system with one or more pieces of equipment for performing polynucleotide analysis (e.g., HPLC, mass spectrometry). The systems may include, for example, detectors for quantifying the analytes via HPLC or mass spectrometry. These systems, or the constituent components thereof, may comprise computational components for performing the analysis, including suitable hardware and software as is known in the art. Various software is available for performing polynucleotide analysis, as is described, for example, in Vanhinsbergh, et al., Anal Chem. 2022 May 24; 94(20):7339-7349 (doi: 10.1021/acs.analchem.2c00765), which is herein incorporated by reference in its entirety. The systems may comprise processors operably connected to memory for performing the analysis. In various implementations, the systems may include processors configured to map the fragments to a reference RNA sequence based, at least in part, on output received from one or more detectors and the target cleavage site(s). In some implementations, the system may be configured to output or provide candidate RNA motif targets for a given reference sequence based on the availability of one or more nucleases (e.g., sequence-specific duplex-dependent nucleases of RNA). The system may allow user-selection of one or more candidate motifs for cleavage. The system may automatically predict the sequences and sizes of fragments resulting from a selected selection of candidate motifs. The system may provide information about the distribution of sizes and/or whether the fragment sizes satisfy any predetermined criteria, as described elsewhere herein. The system may be configured to recommend specific cleavages based on a predetermined availability of nucleases. The system may provide recommended oligonucleotide sequences for performing sequence-specific duplex-dependent cleavages as described elsewhere herein. The system may comprise databases of suitable nucleases (e.g., sequence-specific duplex-dependent nucleases of RNA) and corresponding motifs to automate the selection of cleavage sites and/or nucleases for digestion.

### Example

Digestion of COVID-19 mRNA Vaccine

The Pfizer®-BioNTech® SARS-Cov-2 mRNA vaccine was analyzed for motifs of sequence-specific duplex-dependent nucleases of RNA, specifically the restriction endonucleases TaqI, AvaII, and BanI. Table 3 below indicates the cleavage site of the identified candidate RNA motifs, the specific motif available at each cleavage site, and the restriction endonuclease specific to each.

TABLE 3
TaqI, AvaII, and BanI Restriction Sites within
Pfizer ®-BioNTech ® SARS-Cov-2 mRNA Vaccine.

| mRNA | | |
|---|---|---|
| Cleavage | | |
| Site | Motif | Nuclease |
| 23 | G/GψCC | AvaII |
| 210 | G/GACC | AvaII |
| 268 | G/GCACC | BanI |
| 277 | G/GCACC | BanI |
| 290 | ψ/CGA | TaqI |
| 373 | G/GCACC | BanI |
| 557 | ψ/CGA | TaqI |
| 585 | G/GACC | AvaII |
| 644 | ψ/CGA | TaqI |
| 835 | G/GψGCC | BanI |
| 901 | G/GCACC | BanI |
| 1445 | ψ/CGA | TaqI |
| 1598 | ψ/CGA | TaqI |
| 1855 | G/GCACC | BanI |
| 2152 | G/GCGCC | BanI |
| 2507 | ψ/CGA | TaqI |
| 2511 | G/GACC | AvaII |
| 2725 | G/GCGCC | BanI |
| 2965 | G/GCGCC | BanI |
| 3006 | G/GACC | AvaII |
| 3032 | ψ/CGA | TaqI |
| 3546 | G/GACC | AvaII |
| 3602 | ψ/CGA | TaqI |
| 3647 | ψ/CGA | TaqI |
| 3821 | ψ/CGA | TaqI |
| 3881 | ψ/CGA | TaqI |
| 3931 | G/GψACC | BanI |
| 3957 | G/GψCC | AvaII |

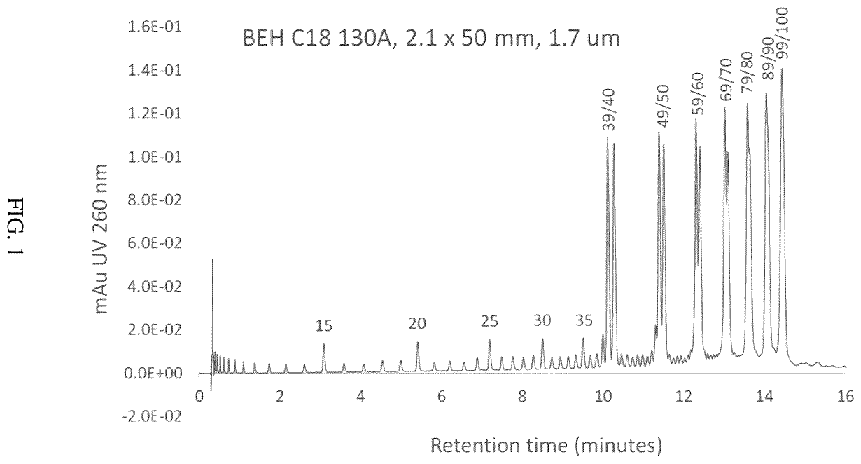

Various digestion schemes were simulated by selecting certain candidate RNA motifs for cleavage and simulating the separation of the resulting digestion fragments by liquid chromatography in silico. Initially, a ladder of 15-100 mer oligodeoxythymidines was chromatographically separated using ion-pairing reversed-phase liquid chromatography (IP RP LC) on a BEH C18 130 Å column (2.1×50 mm, 1.7 μm, available from Waters Corporation, Milford, MA) under the following conditions: column temperature 60° C.; flow rate 0.4 ml/min; run time 20 min; gradient 90-50% A/10-50% B (A=25% acetonitrile, 75% 0.1M HAA pH 8.13; B=75% acetonitrile, 25% 0.1M HAA pH 8.13). Results are shown in FIG. 1. As can be seen, the larger oligonucleotides were more difficult to selectively separate than the smaller oligonucleotides. A calibration curve for predicting retention time (Tᵣ) under these conditions based on oligonucleotide length (L) was fitted to this experimental data as follows:

Tᵣ=b/L²+c/L+d (Formula I),

wherein b=2,450; c=−361; and d=17.77. Using this calibration curve, the separation of oligonucleotide fragments from various hypothetical digestions of the mRNA vaccine were modeled in silico.

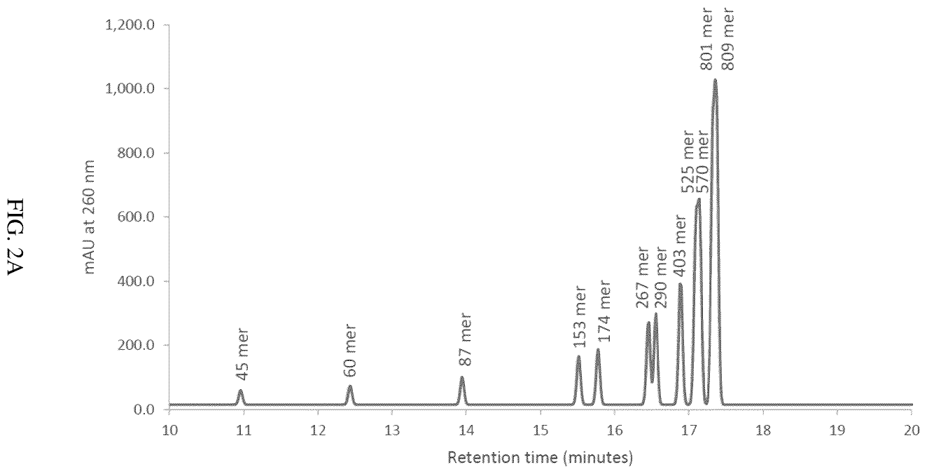

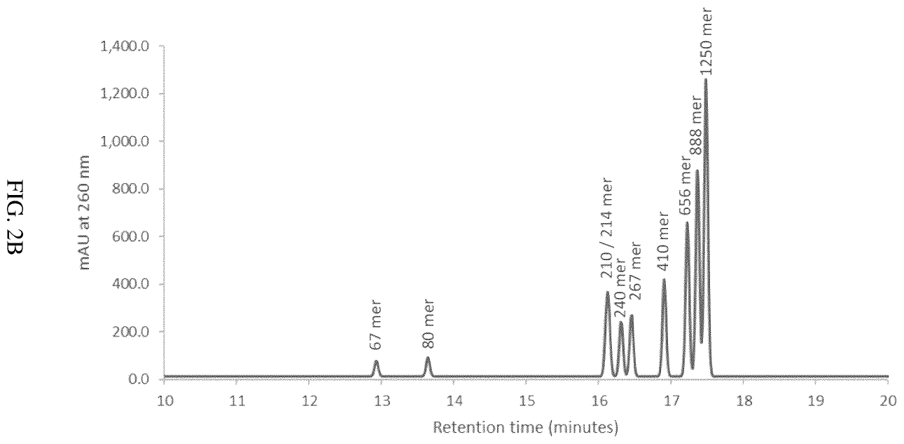

Table 4 below depicts the fragments expected to result from digesting each of the candidate TaqI RNA motifs with TaqI and the expected retention time of each. A simulated chromatogram resulting from the TaqI digestion is shown in FIG. 2A. As seen in FIG. 2A, the 525/570 mer fragments as well as the 801/909 mer fragments were coeluted. It will be understood that a longer column and/or optimized separation method should be able to improve the separation of these fragments. In some implementations, these peaks may be fraction-collected and subjected to additional separation, optionally with additional digestion (e.g., via other restriction endonucleases and/or ribonucleases).

TABLE 4
Digestion of COVID-19 mRNA Vaccine with TaqI Restriction Sites

| Digest | | Retention |
|---|---|---|
| Fragment | Length | Time (min) |
| 5′-290 | 290 | 16.55 |
| 290-557 | 267 | 16.45 |
| 557-644 | 87 | 13.94 |
| 644-1445 | 801 | 17.32 |
| 1445-1598 | 153 | 15.52 |
| 1598-2507 | 909 | 17.38 |
| 2507-3032 | 525 | 17.09 |
| 3032-3602 | 570 | 17.14 |
| 3602-3647 | 45 | 10.96 |
| 3647-3821 | 174 | 15.78 |
| 3821-3881 | 60 | 12.43 |
| 3881-3′ | 403 | 16.89 |

Table 5 below depicts the fragments expected to result from an alternative digestion scheme in which only select RNA motifs are cleaved with TaqI, AvaII, and BanI, and the expected retention time of each. A simulated chromatogram resulting from the digestion is shown in FIG. 2B. As seen in FIG. 2B, peaks corresponding to most of the fragments are distinguishable from one another.

TABLE 5
Digestion of COVID-19 mRNA Vaccine with
Select TagI, AvaII, and BanI Restriction Sites

| Digest | | Retention |
|---|---|---|
| Fragment | Length | Time (min) |
| 5′-210 | 210 | 16.11 |
| 210-290 | 80 | 13.64 |
| 290-557 | 267 | 16.45 |
| 557-1445 | 888 | 17.37 |
| 1445-1855 | 410 | 16.90 |
| 1855-2511 | 656 | 17.23 |
| 2511-2725 | 214 | 16.14 |
| 2725-2965 | 240 | 16.31 |
| 2965-3032 | 67 | 12.93 |
| 3032-3′ | 1252 | 17.48 |

1. A method of digesting an RNA molecule having a known reference sequence into smaller RNA fragments, the method comprising:
forming one or more oligonucleotide duplexes with the RNA molecule along specific portions of the reference sequence;
digesting the RNA molecule into the fragments with one or more sequence-specific nucleases that cleave the RNA molecule at a plurality of predetermined sequence-specific sites, wherein the one or more sequence-specific nucleases comprise one or more duplex-dependent nucleases that only act on RNA within a duplex, and wherein each of the one or more duplexes formed with the RNA molecule comprises a motif recognized by one of the one or more duplex-dependent nucleases.

2. The method of claim 1, wherein the one or more sequence-specific nucleases comprises a plurality of nucleases.

3. The method of claim 2, wherein the plurality of nucleases, comprises a plurality of duplex-dependent nucleases.

4. The method of claim 1, wherein the one or more duplex-dependent nucleases comprises one of more of a restriction endonuclease, a Cas protein, an artificial site-specific RNA endonuclease (ARSE), an enzyme comprising an RNase III domain, or a deoxyribozyme.

5. The method of claim 4, wherein the one or more duplex-dependent nucleases comprises one or more restriction endonucleases selected from the group consisting of AvaII, AvrII, BanI, TaqI, HinfI, and HAEIII.

6. The method of claim 1, wherein the one or more sequence-specific nucleases comprises one or more of RNase T1, RNase A, Colicin E5, and MazF.

7. The method of claim 1, wherein the RNA molecule has a length greater than about 1,000 mers.

8. The method of claim 1, wherein the RNA fragments are between about 10 to 1,000 mers in length.

9.-13. (canceled)

14. The method of claim 1, wherein each of the one or more duplexes is formed with the RNA molecule and another oligonucleotide that is between about 10 and 50 mers in length.

15. The method of claim 1, wherein each of the one or more duplexes is formed with DNA oligonucleotides or by hybridizing an exogenous oligonucleotide with the RNA molecule.

16. (canceled)

17. The method of claim 1, wherein at least one of the sequence-specific nucleases is immobilized on a solid support.

18. The method of claim 17, wherein the at least one immobilized nuclease is provided in the form of an immobilized enzyme reactor (IMER) that allows flow-through digestion of the RNA molecule.

19. The method of claim 18, wherein the nuclease immobilized within the IMER is not a duplex-dependent nuclease and is used to further digest a selected fraction of the RNA fragments after digestion with a duplex-dependent nuclease.

20. The method of claim 1, wherein the RNA molecule is an mRNA molecule and the plurality of predetermined sequence-specific sites comprises a site within about 100 nucleotides of a proximal end of a 3′ poly(A) tail and/or a site within about 100 nucleotides of a 5′ cap.

21. The method of claim 1, further comprising separating one or more of the RNA fragments based on length using liquid chromatography.

22. The method of claim 1, further comprising measuring the mass of one or more of the RNA fragments using mass spectrometry.

23. The method of claim 1, further comprising mapping the RNA fragments to the reference sequence.

22.-29. (canceled)

30. A system for mapping RNA fragments to a reference sequence, the system comprising:
a detector configured to quantify amounts of RNA oligonucleotides between about 20 and 1,000 mers in length; and
a processor operably connected to the detector, wherein the processor is programmed to map detected RNA oligonucleotides to a reference sequence of an RNA molecule based at least in part on the length or mass of the RNA oligonucleotides, wherein mapping the detected RNA oligonucleotides to the reference sequence comprises determining the length of fragments expected to be produced by digesting the RNA molecule into smaller fragments according to claim 1.

31. The system of claim 30, wherein the processor is further configured to automatically identify motifs within the reference sequence for which cleavage with the sequence-specific nucleases would result in fragments between about 20 and 1,000 mers in length, wherein the sequence-specific cleavages comprise one or more selective cleavages with the one or more duplex-dependent nucleases.

32. The system of claim 30, wherein the processor is operably connected to one or more databases comprising a plurality of sequence-specific nucleases and motifs corresponding to each of the sequence-specific nucleases.
